(19) United States
(12) Patent Application Publication
(10) Pub. No.: US 2010/0028432 A1
(43) Pub. Date: Feb. 4, 2010

(54) FORMULATIONS OF ACTIVE PRINCIPLES INCORPORATED IN SLNS SUITABLE FOR TRANSDERMAL ADMINISTRATION

(75) Inventor: Maria Rosa Gasco, Torino (IT)

Correspondence Address: KNOBBE MARTENS OLSON & BEAR LLP, 2040 MAIN STREET, FOURTEENTH FLOOR, IRVINE, CA, 92614, US

(73) Assignee: NANOVECTOR S.R.L., TORINO (IT)

(21) Appl. No.: 12/444,388

(22) PCT Filed: Oct. 5, 2007
(86) PCT No.: PCT/IB07/02971

§ 371 Date: Apr. 3, 2009

(30) Foreign Application Priority Data
Oct. 6, 2006 (IT) MI2006A001918

(51) Int. Cl. A61K 9/10; A61K 9/50; A61K 31/435; A61K 31/405

(52) U.S. Cl. 424/484; 424/502; 514/284; 514/415

(57) ABSTRACT

The present invention relates to formulations suitable for transdermal administration characterized by containing SLNs which contain active principles with a very short half-life and/or drugs with high activity.

### FIELD OF THE INVENTION

The present invention relates to formulations suitable for transdermal administration containing active principles incorporated in SLNs. STATE OF THE ART

There are many drugs which, though very active, exhibit unfavourable kinetics in that their elimination half-life is very short.

Melatonin and apomorphine for example belong to this class.

Melatonin (N-acetyl-5-methoxytryptamine, MT) in particular, for example, is a hormone produced at night by the pineal gland and is concerned with regulating sleep and the circadian rhythm. The synthesis and secretion of MT is induced by darkness and suppressed by light which, by way of the optic nerve fibres, projects onto the cervical ganglion and consequently onto the pineal gland. Plasma levels of MT are low during the day, around 10 pg/ml in a young adult, later increasing sharply at 9.00 pm and reaching a maximum of 70-100 pg/ml between 2.00 and 4.00 am, then returning to low levels between 7.00 and 9.00 am. With ageing, in healthy individuals affected by insomnia, nocturnal MT levels are lower and delayed by an hour compared to those of a young adult with good sleep quality. Administration of MT could be a valid remedy in the treatment of sleep-wake cycle disorders and insomnia. For clinical purposes the administration of exogenous MT should stimulate endogenous MT levels, but this does not occur due to the very short half-life of this substance. When administered by an intravenous bolus injection the half-life of MT is very short (around 40 minutes) and, when administered orally, exhibits a low and variable bioavailability and a rapid plasma clearance influenced by a marked first-pass hepatic metabolism.

U.S. Pat. No. 5,508,039 describes transdermal systems containing melatonin as the active principle in the presence of an absorption enhancer. In the preparation stage of said transdermal systems, melatonin is used as such in the form of a powder.

In vitro tests have demonstrated that, with these transdermal systems, the half-life of melatonin is 2 hours at the most.

Apomorphine also has an elimination time of about 33 minutes.

The need was therefore felt to provide formulations able to considerably extend the half-life of drugs such as the aforementioned.

EP 526666 describes a technique that allows the preparation of solid lipid nanospheres (SLNs) which incorporate active principles.

These types of nanoparticles present a series of advantages, for instance they can cross the mucosa and the blood-brain barrier as described for example in WO 99/27918, be internalised within cells as described in WO 00/030620, be advantageously used for incorporating drugs for use in the treatment of ophthalmic diseases as described in WO 2004/039351, and be used as vehicles for nucleic acids as reported in WO 2005/120469.

### SUMMARY OF THE INVENTION

The present invention relates to formulations suitable for transdermal administration that also contain an active principle having a short half-life and/or high activity, characterized by containing said active principle incorporated in SLNs.

### DESCRIPTION OF THE FIGURES

FIG. 1 represents plasma concentration of MT at baseline and after administration of MT-SLN-TD whereby the y-axis indicates melatonin concentration in pg/ml and the x-axis indicates time in hours.

### DETAILED DESCRIPTION OF THE INVENTION

The term “drugs with a short half-life” means active principles whose half-life is less than 2 hours, being preferably 1 hour.

For example melatonin and apomorphine belong to this class.

For the purposes of the present invention the term “drugs with high activity” means drugs able to display activity even though present, in transdermally administrable formulations, in very small quantities i.e. generally between 1 and 50 mg/g of formulation.

Drugs belonging to this class are those normally used in transdermal systems such as cortisones and hormones and also drugs such as melatonin and apomorphine which, in addition to having a very high activity have, as aforementioned, a very short half-life.

The term “formulations suitable for transdermal administration” in accordance with the present invention means both those applicable via transdermal systems and those not requiring a transdermal system or device for their application.

The formulations applicable or not applicable by the transdermal devices of the present invention are preferably in the form of gels, more preferably hydrogels.

The transdermal systems, being a further aspect of the present invention, are of conventional type such as bilayer systems consisting of: an outermost protective support layer and an adhesive layer in direct contact with the skin that also contains the active principle reservoir (in this case the formulation of the present invention is contained in this layer), or multilayer transdermal systems comprising an outermost protective support layer, an adhesive layer and a layer that acts as the drug reservoir, this being separated from the adhesive layer by the presence of a middle layer.

The transdermal formulations of the present invention can contain absorption enhancers chosen from the class consisting of C8-C20 saturated or unsaturated fatty acids, glyceryl monooleate and relative mixtures.

Particularly preferred fatty acids are octanoic acid and oleic acid.

A further aspect of the present invention are SLNs containing MT having an average diameter of between 50 and 800 nm and a polydispersion index of between 0.06 and 0.90.

The SLNs of the present invention preferably contain MT at concentrations of between 0.5 and 12.5%.

The SLNs are preferably prepared by a process that comprises the following steps:

a) a surfactant and lipid mixture is prepared and heated to the melting point of the lipid, then melatonin is possibly added,
b) an aqueous mixture of at least one co-surfactant and melatonin is prepared, if this latter is not added in step (a), and is heated to the melting point of the lipid used in step (a),
c) the aqueous mixture prepared in step (b) is added to the mixture prepared in step (a) to obtain a hot microemulsion,
d) the hot microemulsion from step (c) is dispersed in water at a temperature preferably between 0.5 and 4° C. to obtain the SLN dispersion,
e) the aqueous dispersion is washed by tangential ultrafiltration with water or an aqueous medium to which a base is possibly added (such as sodium hydroxide, lysine, diethanolamine),
f) this is possibly followed by lyophilization of the aqueous dispersion derived from step (e) possibly in the presence of a cryoprotector, such as trehalose and lactose.

The lipids used in step (a) are preferably chosen from the class consisting of:
Triglycerides, particularly preferred being trilaurin, tricaprylin, tristearin, tripalmitin, triglycerides of capronic acid.
Diglycerides, more preferably dipalmitin and distearin,
Monoglycerides, more preferably glyceryl monostearate,
C10-C30 aliphatic alcohols, more preferably cetyl alcohol and stearyl alcohol,
C10-C22 fatty acids
Cholesterol and relative esters, more preferably cholesteryl hemisuccinate, cholesteryl butyrate, and cholesteryl palmitate.

The surfactants are preferably chosen from the class consisting of:
Phospholipids, preferably phosphatidylcholine and more preferably lecithin such as egg, soya lecithin and relative hydrogenated forms.
Hexadecyl phosphate
Tween 20, Tween 80, Span 20, Span 40 and Span 60,

The co-surfactants are preferably chosen from the group consisting of:
low molecular weight glycols or alcohols such as butanol, hexanol, hexadiol, isopropanol,
alkylaromatic alcohols, such as benzyl alcohol,
aromatic acids, such as benzoic acid,
bile acid salts such as sodium glycocholate, sodium taurocholate and sodium taurodeoxycholate,

The microemulsion obtained in step (c) preferably contains:
lipids at a concentration between: 3 and 10% by weight on the total composition weight,
surfactants at a concentration between 2 and 9% by weight on the total microemulsion weight,
co-surfactants at a concentration between: 2 and 17% by weight on the total microemulsion weight,
melatonin at a concentration between 2 and 7% by weight on the total microemulsion weight,
water at a concentration between 65 and 80% by weight on the total microemulsion weight.

The volume of the aqueous dispersion obtained in step d) of the process of the present invention is preferably from 2 to 20 volumes per volume of the microemulsion.

Some examples of the preparation of SLNs containing MT and the preparation of the relative formulations that can be administered transdermally together with in vitro and in vivo tests of the transdermal activity of said systems are given by way of non-limiting illustration.

All the percentages given in examples 1-3 to follow are weight/weight.

### Example 1

A hot aqueous mixture of benzoic acid, melatonin and sodium taurocholate is added to a mixture of Epikuron 200 and stearic acid heated to 70° C. and maintained under mild agitation, to obtain an aqueous microemulsion having the following composition.

| | |
|---|---|
| Epikuron 200 | 4.9% |
| Stearic acid | 4.56% |
| Benzoic acid | 4.2% |
| Melatonin | 3.1% |
| Sodium taurocholate | 7.1% |
| Water | 76.2% |

The microemulsion is dispersed in cold water at a temperature of between 2 and 4° C. to obtain an aqueous dispersion having a final volume of 4 volumes/volume of the initial microemulsion. This is followed by washing of the aqueous dispersion by tangential ultrafiltration which can be supplemented with a base such as sodium hydroxide solution, to obtain a slightly acidic pH (4.8-5.5). SLNs with an average diameter of 162 nm and a polydispersion index of 0.140 are obtained. Finally, lyophilization is carried out, possibly adding a cryoprotector such as lactose or trehalose.

### Example 2

Apomorphine hydrochloride and a hot aqueous mixture of ascorbic acid and sodium taurocholate are added to a mixture of Epikuron 200, stearic acid and ascorbyl palmitate heated to 70° C. and maintained under mild agitation, to obtain an aqueous microemulsion having the following composition.

| | |
|---|---|
| Epikuron 200 | 4.7% |
| Stearic acid | 7.2% |
| Ascorbyl palmitate | 0.1% |
| Apomorphine hydrochloride | 2.9% |
| Sodium taurocholate | 13.5% |
| Water (2‰ ascorbic acid) | 71.8% |

The microemulsion is dispersed in cold water (2‰ ascorbic acid) at a temperature of between 2 and 4° C. to obtain an aqueous dispersion having a final volume of 4 volumes/volume of initial microemulsion. This is followed by washing of the aqueous dispersion by tangential ultrafiltration.

SLNs with an average diameter of 91.9 nm and a polydispersion index of 0.206 are obtained.

### Example 3

Melatonin is added to a mixture of molten stearic acid at a temperature higher than 70° C. and Epikuron 200. A solution of sodium taurocholate in water, heated to the same temperature as the aforesaid mixture, is then added slowly and under agitation to the aforesaid mixture.

A transparent microemulsion is thus obtained having the following composition:

| | |
|---|---|
| Epikuron 200 | 3.9% |
| Stearic acid | 7.3% |
| Melatonin | 2.9% |
| Sodium taurocholate | 13.3% |
| Water | 72.6% |

The microemulsion is dispersed in cold water at a temperature of between 2 and 3° C. to obtain an aqueous dispersion having a final volume of 3 volumes/volume of initial microemulsion. This is followed by washing of the aqueous dispersion with water by ultrafiltration.

SLNs with an average diameter of 91 nm and a polydispersion index of 0.135 are obtained.

Finally, lyophilization is carried out, possibly adding a cryoprotector such as lactose or trehalose.

### Example 4

### A) Preparation of SLNs

Using the same method as given in the preceding examples, SLNs containing melatonin were prepared having the following characteristics: MT content in SLNs 1.8%, average diameter 85 nm, polydispersion index 0.135.

### B) Preparation of the Gel, Results of In Vitro Tests and Discussion

A gel was prepared in accordance with the following method: 200 mg of SLNs with the characteristics given in (A) were added under mild agitation to 2 g of a mixture (w/w) of water and propylene glycol (70:30) to which Carbopol® 940 was subsequently added, while continuing to agitate, at concentrations such that the final concentration of said component within the formulation was 1.20% by weight, to hence obtain a gel. This gel was used to conduct in vitro experiments in accordance with the following operating methods:

Vertical cells of Franz type were used: the dorsal skin of athymic nude mice, 4-5 weeks of age, were used. 0.3 g of the gel were deposited on a suitably washed 1.6 cm² diffusion area of the skin. The contents of said receptor cell were continuously stirred and the temperature controlled at 25° C. At specific intervals the contents of the receptor chamber were removed for HPLC determination and the cell was refilled with fresh aqueous solution at pH 6.0. The experiments were carried out in triplicate.

A melatonin flow of 1 μg/h/cm² across hairless mouse skin and with pseudo zero order kinetics was observed.

The pseudo zero order kinetics of melatonin incorporated in SLNs has prompted us to study, in particular, the transdermal in vivo release of melatonin incorporated in SLNs.

### Example 5

### A) Preparation of SLNs

Using the same method as given in the preceding examples, SLNs containing melatonin were prepared having the following characteristics: MT content in SLN 2%, average diameter 91 nm, polydispersion index 0.140.

### B) Preparation of Transdermal Gel

The SLNs having the aforesaid characteristics were dispersed in a mixture of water and propylene glycol (70/30) to which Carbopol 940® was then added at concentrations such that the quantity by weight of said component was 1.20% by weight, to obtain a gel (MT-SLN-TD) whose melatonin concentration was 1.8 mg/g.

### C) In Vivo Tests

2 g of MT-SLN-TD prepared as described in (B) were applied to 9 cm² of skin on the upper thorax of 10 subjects (5 males and 5 females, average age 48.2±16.3 years) at 8.30 am.

The area of skin on which the MT-SLN-TD gel was applied as a single layer of 1 mm thickness directly in contact with the skin surface, which could hence function as a MT reservoir, was delimited by double adhesive tapes of the same thickness then covered with a polyester membrane (3M SCOTCHPAK®) and with an occlusive membrane (Smith and Nephew Opsite Flexigrid®) with the purpose of preventing evaporation of some of the components. The skin was not pre-treated. This type of patch was removed after 24 hours. Blood samples were collected from all the subjects at 9.00 am, i.e. half an hour after administration, thereafter every 2 hours up to eleven hours following administration. As controls for baseline determination, blood samples were collected 1 week after administration and at the same time intervals from 9.00 am until 7.00 pm.

Over the next 2 days, blood samples from two of the subjects were taken every 3-4 hours from 8.00 am to 7.00 pm on the second day and every 3-4 hours from 8.00 am to 3.00 pm on the third day.

### D) Determining Concentration of MT in Plasma

The withdrawn blood was centrifuged and stored at −20° C. until analysis. The plasma concentration of MT was determined by extraction with ethyl ether according to the RIA method.

All samples deriving from the same patient were analysed with the same kit. The sensitivity of the analysis was 2 pg/ml. Duplicate determinations were carried out for each sample.

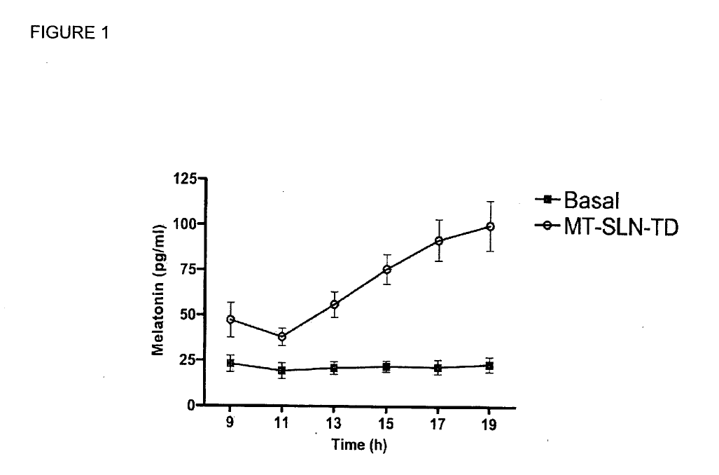

As shown in FIG. 1 the plasma concentration of MT, obtained after MT-SLN-TD administration, was found to be greater than the baseline concentration.

In this respect an increase over time was attained, passing from a basal value of 25 pg/ml to more than 90/100 pg/ml.

Also, in the two subjects from whom further blood withdrawals were made for over 50 hours, it could be demonstrated that plasma MT levels far higher than basal values can be obtained for more than 24 hours, the maximum concentration being reached after 12-18 hours depending on the subject.

1. Formulations suitable for transdermal administration comprising solid lipid nanoparticles (SLNs) incorporating at least one active principle having a short elimination half-life.

2. Formulations as claimed in claim 1 further comprising absorption enhancers, preferably selected from the group consisting of: C8-C20 saturated or unsaturated fatty acids, glyceryl monooleate and relative mixtures.

3. Formulations as claimed in claim 1, wherein said short half life active principles are melatonin or apomorphine.

4. Formulations as claimed in claim 3 in a gel form.

5. Formulations as claimed in claim 4 in a hydrogel form.

6. Transdermal systems comprising the formulation claimed in claim 1.

7. Solid lipid nanoparticles (SLNs) incorporating melatonin (MT) or apomorphine.

8. SLNs as claimed in claim 7 having an average diameter of from 30 to 800 nm and a polydispersion index of from 0.06 to 0.90.

9. SLNs as claimed in either of claims 7 or 8 wherein melatonin (MT) is in concentrations from 0.5 to 12.5%.

10. Process for preparing SLNs as claimed in claim 7, comprising the following steps:
a) preparing a surfactant and lipid mixture by heating said mixture to the melting point of the lipid, optionally adding melatonin or amorphin,
b) preparing an aqueous mixture of at least one co-surfactant and melatonin or apomorphin, if melatonin or amorphin are not added in step (a), and heating to the melting point of the lipid used in step (a),
c) adding the aqueous mixture prepared in step (b) to the mixture prepared in step (a) to obtain a hot microemulsion,
d) dispersing the hot microemulsion from step (c) in water at a temperature preferably between 0.5 and 4° C. to obtain the SLN dispersion,
e) washing the aqueous dispersion by ultrafiltration with waters optionally adding a base thereto, and
f) optionally lyophilizing the aqueous dispersion derived from step (e) possibly in the presence of a cryoprotector.

11. Process as claimed in claim 10 wherein said lipids are chosen from the group consisting of: triglycerides, diglycerides, monoglycerides, C10-C30 aliphatic alcohols, C10-C22 fatty acids and relative esters, cholesterol and relative esters.

12. Process as claimed in claim 10, wherein the surfactants in step (a) are chosen from the group consisting of phospholipids, hexadecyl phosphate, Tween and Span.

13. Process as claimed in claim 11, wherein or the co-surfactants are chosen from the group consisting of: glycols or alcohols, low molecular weight fatty acids, alkylaromatic alcohols, aromatic acids and bile acid salts.

14. Process as claimed in claim 11, wherein the microemulsion obtained in step (c) contains lipids at a concentration of from 3 to 10% on the total microemulsion weight, surfactants at a concentration of from 2 to 9% by weight on the total microemulsion weight, co-surfactants at a concentration of from 2 to 17% by weight on the total microemulsion weight, melatonin at a concentration of from 2 to 7% by weight on the total microemulsion weight and water at a concentration of from 60 to 80% by weight of the total microemulsion weight.

15. Process as claimed in claim 10, wherein the volume obtained in step (d) of the process of the present invention is from 2 to 20 volumes per volume of microemulsion.
